(19) United States
(12) Patent Application Publication
(10) Pub. No.: US 2008/0312595 A1
(43) Pub. Date: Dec. 18, 2008

(54) ARTICULATED ACTUATOR FOR IMPLANTABLE PUMP

(75) Inventors: Ahmed Elmouelhi, Minneapolis, MN (US); James M. Haase, Maplewood, MN (US); Ronald L. Mezera, Lake Elmo, MN (US); Nicholas R. Whitehead, Hopkins, MN (US)

Correspondence Address: MEDTRONIC, INC., 710 MEDTRONIC PARKWAY NE, MINNEAPOLIS, MN, 55432-9924, US

(73) Assignee: Medtronic, Inc.

(21) Appl. No.: 11/761,673

(22) Filed: Jun. 12, 2007

(51) Int. Cl. A61M 1/00

(52) U.S. Cl. 604/151

(57) ABSTRACT

The present invention is an actuator for a piston pump that includes a separately formed piston and armature. The piston and the armature are later assembled together or are inserted into the piston pump in such a manner as to cooperate during pumping. Assembling the piston and the armature as separate components may provide for improved form of the piston component when manufactured separately from the armature, due to, for example, increased simplification of the manufacturing process. In addition, effects of manufacturing the piston and the armature together, such as stress on the piston, may be reduced.

### FIELD

This invention relates generally to a drug pump. More particularly, the present invention relates to a multiple piece actuator for a drug pump.

### BACKGROUND

Infusion devices may be used to deliver an infusion media (e.g. a medication such as insulin) to a patient. Such devices may be designed to be implanted into a patient's body to deliver predetermined dosages of the infusion media to a particular location within the patient's body, e.g. in the venous system, the spinal column, or within the peritoneal cavity. A known infusion device of the type described above includes a drive mechanism that includes a reciprocating pumping element, otherwise known as an actuator member. The reciprocating pumping element includes an actuator that has a piston portion coupled to an armature portion, also known as a piston actuator or pole. The piston portion is configured to reciprocate within a piston channel when a solenoid coil is alternately energized and de-energized. That is, when the solenoid is energized, magnetic flux causes the actuator to move very quickly (i.e. in the order of 2-3 milliseconds) until it reaches a stop member. This corresponds to the pump's forward stroke and results in the delivery of a predetermined dosage of infusion media from an outlet chamber to the patient. When the solenoid is de-energized, the lack of magnetic flux allows the actuator to return to its original position under the force of a spring or other return mechanism. This, in turn, causes the pressure in the piston chamber to fall. The reduced pressure in the piston chamber causes infusion media to flow from a reservoir through an annulus between the actuator piston and the piston cylinder wall to refill the piston chamber, thus equalizing the pressure between the reservoir and the piston chamber and preparing the pump for its next pumping or delivery stroke.

Manufacturing of the actuator as a single piece made up of the piston portion and the armature portion requires near perfect tolerances in form and perpendicularity to assure the trouble free operation of the actuator over the life of the pump. If the perpendicularity of the piston from the armature is off slightly, or the piston is not located correctly on the armature, wear issues may develop that could cause premature failure of the pump. A continued need therefore exists for actuator designs that reduce manufacturing costs and improve performance.

### BRIEF SUMMARY OF THE INVENTION

According to an aspect of the invention, a

more

### BRIEF DESCRIPTION OF THE DRAWINGS

Embodiments of the present invention will hereinafter be described in conjunction with the following drawings wherein like reference numerals denote like elements throughout.

FIG. 1 is an isometric view of an implantable infusion device in accordance with one embodiment of the present invention.

FIG. 2 is a representative view of an infusion device implanted into a body of a patient in accordance with one embodiment of the present invention

FIG. 3 is a cross-sectional view of a drive mechanism in accordance with a first embodiment of the present invention.

FIG. 4 is an exploded view of an embodiment of the drive mechanism shown in FIG. 3.

FIG. 5A is an exploded perspective view of one embodiment of an actuator member of the present invention.

FIG. 5B is a perspective view of the embodiment shown in FIG. 5A.

FIG. 6A is an exploded perspective view of another embodiment of an actuator member of the present invention.

FIG. 6B is an exploded perspective view of the embodiment shown in FIG. 6A.

FIG. 7A is an exploded perspective view of another embodiment of an actuator member of the present invention.

FIG. 7B is an exploded side view of the embodiment shown in FIG. 7A.

### DETAILED DESCRIPTION OF THE INVENTION

The present invention is an actuator for a piston pump that includes a separately manufactured piston and armature. The piston and the armature are later assembled together or are inserted into the piston pump in such a manner as to cooperate during pumping. Assembling the piston and the armature as separate components may provide for improved form of the piston component when manufactured separately from the armature, due to, for example, increased simplification of the manufacturing process. In addition, effects of manufacturing the piston and the armature together, such as stress on the piston, may be reduced. Moreover, as further described below, allowing for a joint or point surface contact between the piston and the armature may reduce or eliminate the ability of the pole to induce wear on the armature, or visa versa, through stresses or through rotation of the components during the pumping process. The armature and piston may also be referred to other names without effecting the scope of the present invention, such as, for example, the armature may be known as a pole

In one embodiment, the actuator is formed as three pieces and assembled before placement in the pump. In further embodiments the actuator may be separately formed in two or more pieces and then assembled before or after placement of one or more pieces into the pump. As may be appreciated, the separate pieces of the actuator may be connected in a number of ways, including friction fitting, snapping, welding, sonic welding, soldering, gluing, etc. In further embodiments no permanent connection is necessarily be established between the piston and the armature. The term “assembled” should therefore not be limited to making a permanent or locking type engagement of the pieces of the actuator but instead may only imply arrangement of the pieces in a manner designed to perform the actuator function.

The following detailed description is of the presently contemplated mode of implementing the invention. This description is not to be taken in a limiting sense, but is merely for the purpose of illustrating the general principles of embodiments of the invention. Furthermore, there is no intention to be bound by any theory presented in the preceding background of the invention or the following detailed description of the invention. The scope of the invention is defined by the appended claims.

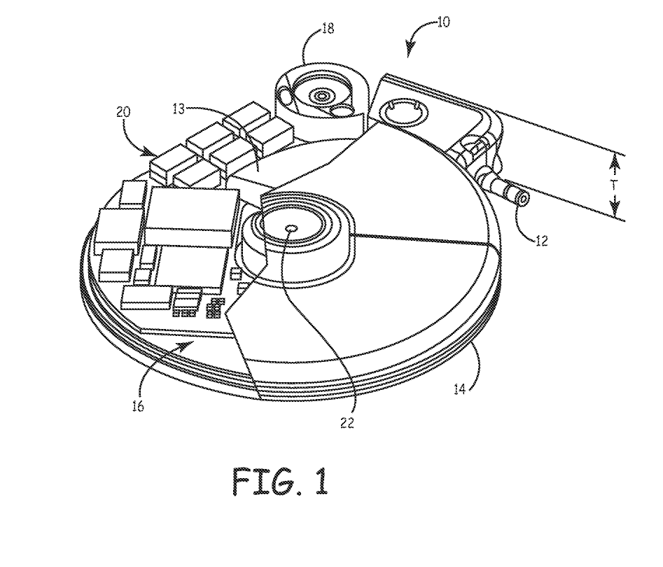

FIG. 1 shows an implantable infusion device 10. The illustrated device 10 is configured to be surgically implanted into a patient, for example, in the abdominal region, between the skin and the abdominal wall. A catheter (not shown) connected to the infusion device 10 may deliver infusion medium to the patient, for example, but not limited to, by feeding infusion medium to a particular location in the venous system, within the spinal column, or in the peritoneal cavity of the patient. Other embodiments of the infusion device 10 may be implemented as external infusion devices that connect to patients through suitable catheter devices or the like. Yet further embodiments of the infusion device 10 may be used in other contexts, e.g., for delivery of a medium into other suitable environments. Therefore, for purposes of simplifying the present disclosure, the term “patient” is used herein to refer to any environment in which an implantable device is implanted or to which an external device is connected, whether or not the implant or connection is carried out for medical purposes. Also, the term “infusion medium” is used herein to refer to any suitable medium delivered by the drive device.

In further embodiments, the present invention actuator member may be included in pumping systems not related to infusion devices. An implantable infusion pump, however, will be utilized in the remainder of this description for the sake of simplicity.

A description of the implantable infusion pump and how it is placed in the body may help to provide some further context for the present invention. The device 10 may include a generally disc-shaped housing 14. While a generally circular disc-shaped embodiment is illustrated in FIG. 1, it will be understood that further embodiments of the infusion device 10 may employ housing of other shapes, including, but not limited to, oval, oblong, rectangular, or other curved or polygonal shapes. Generally, the housing 14 is made of a biocompatible material and most often has a relatively small diameter and thickness to reduce patient trauma during implant surgery and after implantation.

The housing 14 includes a reservoir 16 for holding a volume of infusion medium, such as, but not limited to, a liquid medication to be administered to the patient. Housing 14 may also contain a drive mechanism 18 (e.g. a pump), a power source 13, and control electronics 20. Pump 18 may be configured to receive infusion media from reservoir 16 via a pump inlet 22. Inlet structure 22 may provide a closeable and sealable fluid flow path to the reservoir 16 in the reservoir portion of the housing. The inlet structure 22 may include a port for receiving a needle through which fluid may be transferred to the infusion device, for example, to fill or re-fill the reservoir 16 of the device with the infusion media or a rinsing fluid as will be more fully discussed below. In particular embodiments, the inlet structure 22 may be configured to re-seal after a fill or re-fill operation, and to allow multiple re-fill and re-seal operations. One example of an inlet structure 22 is described in U.S. Pat. No. 6,652,510, titled “Infusion Device and Reservoir for Same,” which is incorporated by reference herein in its entirety and for everything it teaches and discloses. However, further embodiments may employ other suitable inlet structures, including, but not limited to, those described in U.S. Pat. Nos. 5,514,103 and 5,176,644, each to Srisathapat et al.; U.S. Pat. No. 5,167,633 to Mann et al.; U.S. Pat. No. 4,697,622 to Swift; and U.S. Pat. No. 4,573,994 to Fischell et al., also incorporated by reference. Representative examples of reservoir housing portions and reservoirs which may be employed in embodiments of the invention are described in the above referred to U.S. Pat. No. 6,652,510, and further embodiments may employ other suitable reservoir configurations, including, but not limited to, those described in the above referred to U.S. Pat. Nos. 5,514,103; 5,176,644; 5,167,633; 4,697,622; and 4,573,994.

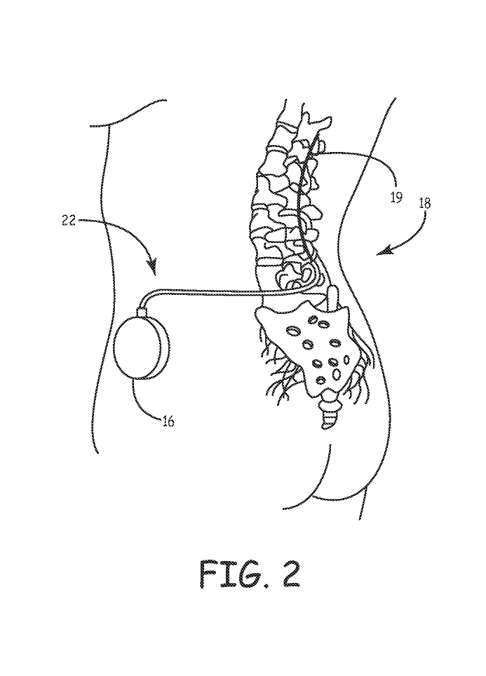

FIG. 2 may illustrate an example placement of one embodiment of an implantable infusion system that is implanted within a patient's body 15. The exemplary infusion systems depicted in implantable medical device 10, and preferably at least one catheter 12. Such infusion systems may be used for a wide variety of therapies including treatment of pain, spasticity, and other medical conditions. Although exemplary infusion systems that may be used in connection with the present invention are described herein, reference may also be had to U.S. Patent Application Publication No. US 2005/0075624 A1, titled

Pressure Sensing in Implantable Medical Devices (Miesel), which describes infusion systems that may be modified for use accordance with the methods of the present invention.

The medical device 10 and catheter 12 are typically implanted by a clinician (e.g., surgeon) within the body 15 during a surgical procedure. While the present invention also contemplates embodiments wherein the catheter is implanted with a proximal end outside the body 15 so that it may attach to an external infusion device, the remainder of this description is, for the sake of brevity, directed to implantable infusion systems that are entirely implanted in the body 15 of the patient.

Before implantation of the medical device 10, the catheter 12 may be positioned such that the fluid delivered to the patient through the catheter 12 reaches a selected internal delivery location 17 within the body 15 of the patient. As depicted, the infusion system is implanted such that the delivery site 17 is located within the intrathecal space of the spinal canal. As may be appreciated, the infusion systems of the present invention may be used to deliver fluid to any other selected internal delivery location, e.g., epidural, etc.

Catheter 12 may preferably disgorge fluid at other than at its distal end. For example, catheter 12 may intentionally have a delivery region that is not proximate the distal end of the catheter 12, e.g., a hole or valve positioned somewhere before reaching the distal end of the catheter 12. Thus, catheter 12 may be placed in patient with a delivery region of catheter 12 placed in or near to, generally proximate to, the selected internal delivery site 17.

A proximal end of the catheter 12 may be tunneled through the tissue to the device implant location and coupled to a catheter port of the medical device 10. If implanted, the medical device 10 is typically positioned subcutaneously, e.g., from 1 centimeter (0.4 inches) to 2.5 centimeters (1 inch) beneath the skin, where there is sufficient tissue for supporting the medical device 10, e.g., with sutures or the like.

The medical device 10 is, in the illustrated embodiment, operable to infuse a fluid from an enclosed reservoir 16 into the body 15 through the catheter 12.

In order to fully understand the present invention, a more detailed explanation of an example embodiment pump in which the invention may be utilized may be first helpful.

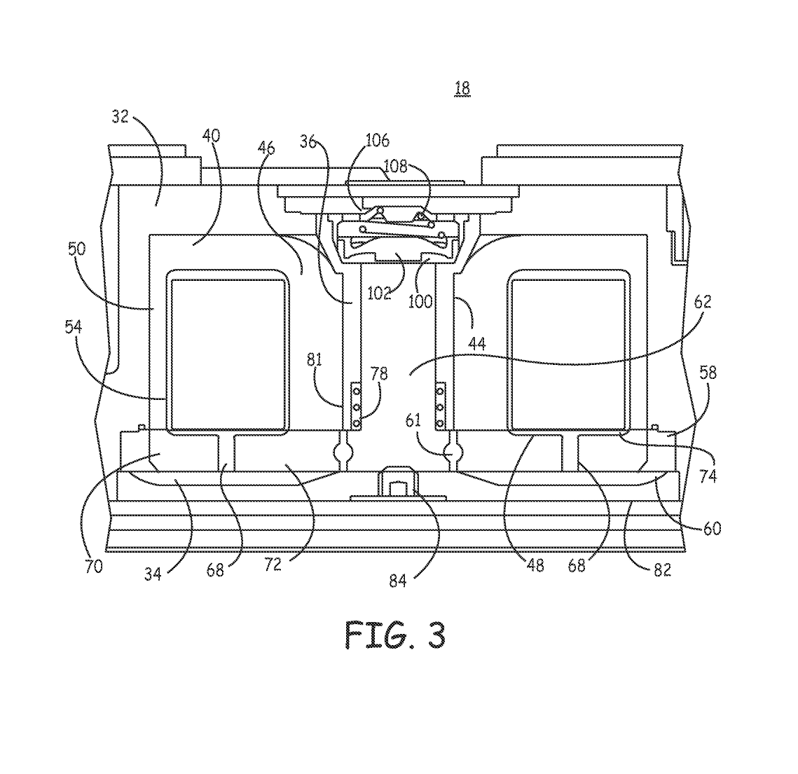

As illustrated in FIG. 3, pump 18 may include an outlet (not shown) through which the infusion medium may be expelled. When the device 10 is implanted in a patient or connected externally to a patient, the catheter 12 may be connected to the outlet to deliver expelled infusion medium into the patient's blood stream or to a selected location in the patient's body. The drive mechanism may be controlled to deliver infusion medium in any suitable manner, for example, according to a programmed dispensing rate or schedule or according to an actuation signal from a sensor, timer or other suitable source.

In particular embodiments, both the drive mechanism 18 and the reservoir 16 may be hermetically sealed. In such embodiments, the housing 14 containing drive mechanism 18 and control electronics 20 may be made from titanium or titanium alloy or other biocompatible metals. The reservoir portion 16 of the housing may be made from similar metals or a biocompatible and infusion medium compatible plastic that allows for the desired hermeticity.

The drive mechanism 18 may include mechanical and electromagnetic components that inhabit a volume of space within the housing 14 in which the components reside and operate. The device 10 is configured such that, once implanted, it functions for a relatively long period of time to administer infusion medium to the patient to periodically be replenished from the outside of patient's body.

As used herein, the term “therapeutic substance” refers to a substance intended to have a therapeutic effect on the patient, e.g., pharmaceutical compositions, genetic materials, biologics, and other substances. “Pharmaceutical compositions,” as used herein, may include chemical formulations intended to have a therapeutic effect such as intrathecal antispasmodics, pain medications, chemotherapeutic agents, and the like. Pharmaceutical compositions are often configured to function effectively in an implanted environment by possessing various characteristics including: stability at body temperature to retain therapeutic qualities; concentration to reduce the frequency of replenishment; and the like. “Genetic materials,” as used herein, may include substances intended to have a direct or indirect genetic therapeutic effect such as genetic vectors, genetic regulator elements, genetic structural elements, DNA, and the like. “Biologics,” as used herein, may include substances that are living matter, or derived from living matter, and offer a therapeutic effect to the patient such as stem cells, platelets, hormones, biologically produced chemicals, and the like. “Other substances” may include most any other substance that is intended to have a therapeutic effect, yet does not clearly fit within one of the categories identified above. Examples of other substances may include saline solutions, fluoroscopy agents, and the like.

In some embodiments, the fluid contained within a reservoir 16 of the medical device 10 may be replenished periodically after device implantation. Typically, replenishment is accomplished with a non-coring needle (not shown) connected to a syringe filled with the fluid. The needle may be inserted through the patient's skin and into a self-sealing septum located within the housing of the medical device 10.

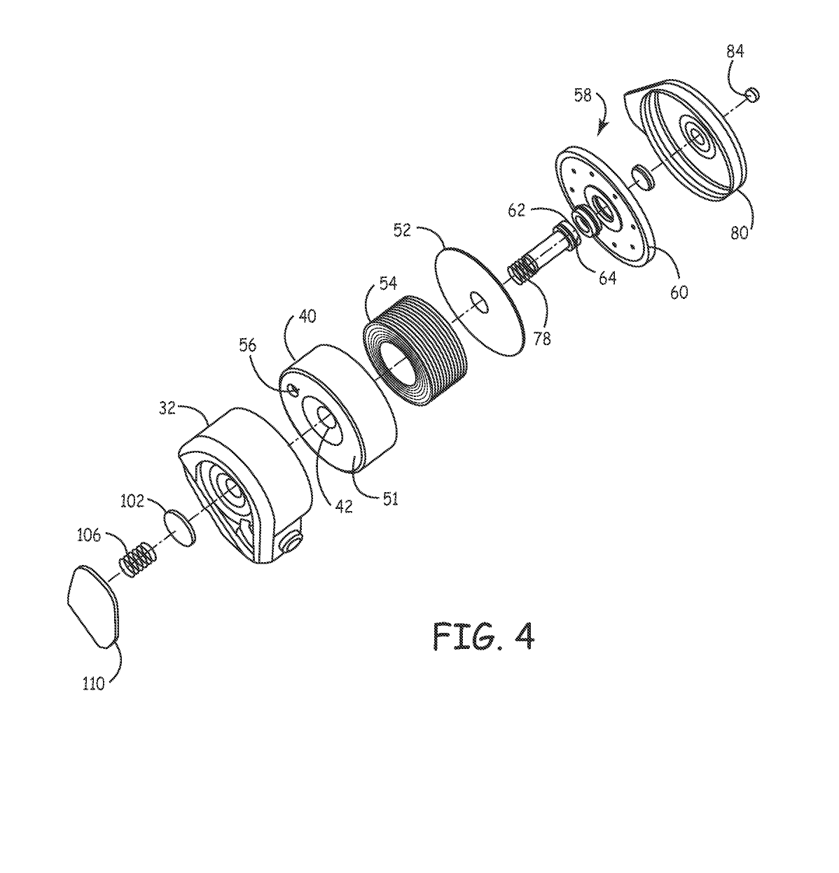

FIG. 3 is a cross-sectional view of a drive mechanism 18 in a retracted position or state. The drive mechanism 18 may employ electromagnetic and mechanical forces to change (or move) between retracted and forward positions or states, also known as first and second positions or states, to cause infusion medium to be drawn in through an inlet and forced out of the outlet, respectively. The assembly of components shown in FIG. 3 is also shown in an exploded view in FIG. 4.

Referring to FIGS. 3 and 4, the drive mechanism 18 may include a housing member 32 that is open on one side to a hollow, annular interior section 34. The housing 32 has a central hub portion 36 with a central piston channel 38. The bottom side of the housing member 32 (with reference to the orientation shown in FIG. 3) includes an opening to the hollow interior section 34 through which coil wires may pass. The bottom side of the housing member may also include a configuration of recesses and cavities for providing an outlet chamber and an outlet passage. The housing member 32 is most often made of generally rigid, biocompatible and infusion medium compatible material having no or low magnetic permeability such as, but not limited to, titanium, stainless steel, bio-compatible plastic, ceramic, glass or the like.

As shown in FIGS. 3 and 4, a coil cup 40 is located within the annular interior section 34 of the housing 32. The coil cup 40 may have a generally cylinder shape, open on one side to a hollow, annular interior. The coil cup 40 may include a bore 42 located in a central hub portion 44 and extending axially relative to the annular interior. The hub portion 44 of the cup member defines an inner annular wall 46 having an end surface 48 (or inner pole surface) having a defined width. The cup member 40 has an outer wall 50 having an end surface 52 (or outer pole surface) having a width. The outer wall 50 is connected to the inner wall 46 of hub portion 44 by a backiron portion 51 of the cup member 40. At the open end of cup member 40 the end surfaces 48 and 52 of the inner and outer walls 46 and 50, respectively, define pole surfaces that cooperate with pole surfaces on an armature to provide a path for electromagnetic flux during a forward stroke of the drive mechanism.

When assembled, the coil cup 40 may be located in the hollow interior of the housing member 32, with the central portion 36 of the housing 32 extending through channel 42 of the coil cup 40 as shown in FIG. 3. A coil 54 may be located within the hollow, annular interior of the coil cup 40 and disposed around the axis of the annular interior of the coil cup 40. The coil cup 40 is provided with an opening 56 through which coil leads extend, as shown in FIGS. 3 and 4. The coil cup 40 may be made of generally rigid material having a relatively high magnetic permeability such as, but not limited to, low carbon steel, iron, nickel, ferritic stainless steel, ferrite, other ferrous materials, or the like. The coil 54 may include a conductive wire wound in a coil configuration. The coil wire may include any suitable conductive material such as, but not limited to, silver, copper, gold or the like, with each turn electrically insulated from adjacent turns and the housing. In one particular embodiment, the coil wire may have a square or rectangular cross-section to achieve minimal space between windings and a greater number of coil turns thus improving electrical efficiency.

The drive mechanism 18 may also include an actuator member 58, which may include an armature portion 60 and a piston portion 62. The actuator member 58 is most often made of a generally rigid, biocompatible and infusion medium compatible material having a relatively high magnetic permeability such as, but not limited to, ferrous materials, ferritic stainless steel with high corrosion resistance, or the like.

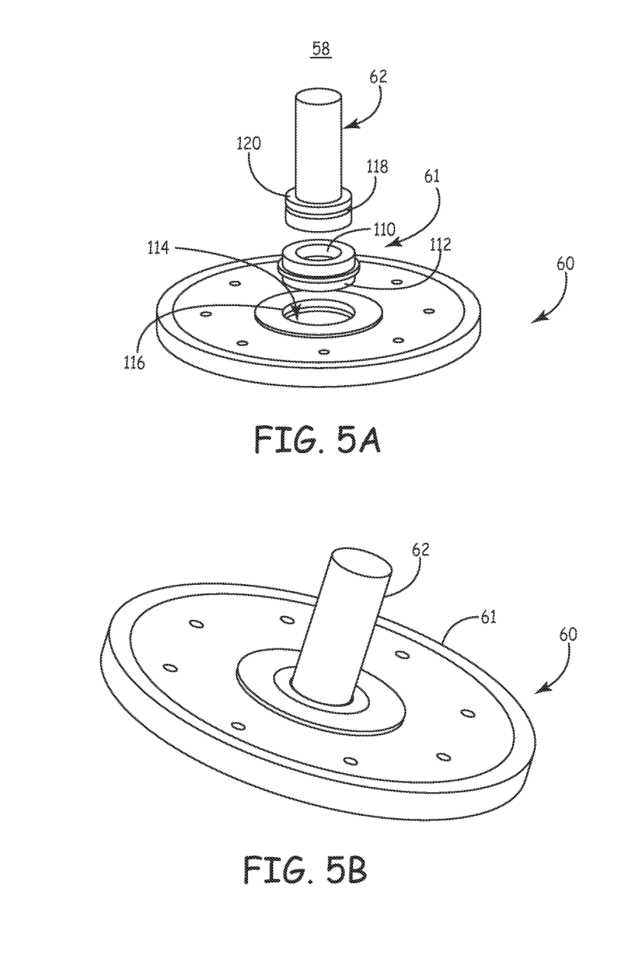

As illustrated in FIGS. 3 and 5A, the actuator member 58 of one embodiment includes an armature portion 60, a piston portion 62, and a collar 61. The piston portion 62 may be joined to the collar 61 which is in turn is joined or attached to the armature portion 60.

The collar 61 may further include an interior rib 110 and an exterior rib 112. The interior and exterior ribs 110 and 112, may be molded into the collar 61 to effectuate placement or friction fitting of the collar 61 into the armature portion 60 and attachment of the piston portion into the collar 61. The collar 61 may be made of any desired material but may be preferably made of a flexible material such as rubber or a pliable plastic. Materials may include natural or synthetic rubber, Neoprene, Teflon, latex, silicon based materials, or the like. In addition, the material should be compatible with whatever liquids are being pumped.

The armature portion 60 in the present embodiment may be a generally flat disk with a receiving portion 114 for receiving the collar 61. The receiving portion 114 is generally formed in the center of the armature portion 60 and may include a groove 116 for receiving the exterior rib 112 of collar 61. The receiving portion 114 in some embodiments may or may not be created through the entire armature portion 60. The receiving portion 114 may also be molded into the armature portion 60.

The piston 62 may also include a groove 118 for securing the piston 62 to the collar 61 through interaction with the interior rib 110. The groove 118 may be formed in shoulder 120 at the end of the piston 62. The piston 62, shoulder 120, and groove 118 may be formed as on unitary structure or as separate structures and attached together. As may be appreciated, the piston 62, collar 61, and armature 60, and the corresponding grooves and ribs thereon, are formed of a size and shape whereby each piece will cooperate in an interlocking fashion with the corresponding pieces. As may be further appreciated, a number of different corresponding shapes may be utilized to effectuate a friction fit of the various components.

Flexible materials for formation of the collar 61 may allow for a non-rigid relationship to exist between the armature portion 60 and the piston portion 62. The collar 61 may be stiff enough to retain the components in a desired position but flexible enough to allow for some movement of the piston 62 relative to the armature 60. Such a flexible relationship between the components may allow for reduced wear in the components and reduced requirements that the pieces be manufactured to relatively narrow manufacturing tolerances.

The armature 60 cooperates with the inner and outer walls of the coil cup 40 to provide a flux path for electromagnetic flux. The spacing between the pole surfaces on the armature 60 and the pole surfaces on the coil cup walls define gaps in the flux path. In particular embodiments, the spacing between the surface of outer pole 70 of the armature 60 and the surface of outer pole 52 of the outer wall 50 of the coil cup 40 is greater than the spacing between the surface of inner pole 72 of the armature and the pole surface 48 of the inner wall 46 of the coil cup (or the barrier 74) when the actuator is in the retracted position shown in FIG. 3.

The radial struts 68 in the armature provide radial paths for electromagnetic flux between outer and inner pole sections 70 and 72 of the armature. The configuration of openings is most often designed to provide a sufficient conductor for electromagnetic flux and yet minimize or reduce viscous resistance to actuator motion. With reference to FIG. 3, the actuator member 58, including the assembled piston 62, armature 60, and collar 61, is arranged with the piston portion 62 extending through the axial channel 38 of the housing 32 and with the armature portion 60 positioned adjacent to the open side of the coil cup 40. An actuator spring 78 may be positioned to force the armature portion 60 of the actuator 58 in the direction away from the open side of the coil cup 40 to provide a gap between the armature 60 and the open side of the coil cup 40. A biocompatible and infusion medium compatible barrier 74 is located over the open side of the coil cup 40 between the armature 60 and the coil cup 40 to help seal the annular interior of the coil cup 40 and coil 54. In other embodiments in which infusion medium may contact the coil, the barrier 74 may be omitted.

The actuator spring 78 in the illustrated embodiment is a coil spring disposed around the piston portion 62 of the actuator 58 adjacent the armature portion 60. One end of the coil spring abuts the armature portion 60 of the actuator, while the opposite end of the coil spring abuts a shoulder 81 in the piston channel 38 of the housing 32. In this manner, the actuator spring 78 imparts a spring force between the housing and the actuator 58 to urge the actuator toward its retracted position shown in FIG. 3. As may be appreciated, the friction fitting or otherwise engagement of the piston 60, collar 61, and armature 62 must be of sufficient strength to allow for the actuator 58 to be moved back and forth in this manner without causing the components to separate.

In the illustrated embodiment, by using a coil spring 78 located around and coaxial with the piston portion 62 and disposed partially within the piston channel 38, the actuator spring may have minimal or no contribution to the overall thickness dimension of the drive mechanism. However, in other embodiments, actuator springs may have other suitable forms and may be located in other positions suitable for urging the actuator toward its retracted position shown in FIG. 3. The actuator spring 78 is most often made of a biocompatible and infusion medium compatible material that exhibits a suitable spring force such as, but not limited to, titanium, stainless steel, MP35N cobalt steel or the like.

The drive mechanism 18 may further include a cover member 80 which attaches to the housing member 32 over the open side of the housing member and the barrier 74. The cover member 80 is most often made of a generally rigid, biocompatible and infusion medium compatible material having a relatively low magnetic permeability (being relatively magnetically opaque) such as, but not limited to, titanium, stainless steel, biocompatible plastic, ceramic, glass or the like.

The cover member 80 defines an interior volume 82 between the barrier 74 and the inner surface of the cover member. The armature portion 60 of the actuator member 58 resides within the interior volume 82 when the cover is attached to the housing. The armature 60 is moveable in the axial direction within the volume 82 between the retracted position shown in FIG. 3 and the forward stroke position, which is described in more detail below. This movement is created by the action of electromagnetic force generated when a current is passed through the coil 54 and the mechanical return action of the actuator spring 78.

An adjusting stop 84, or adjustable plunger, may be located within the cover 80 for contacting the armature 60 when the armature is in the fully retracted position shown in FIG. 3 to set the retracted position of the armature. In particular embodiments, a seal (e.g. a silicon rubber sealing ring) may be disposed between the stop 84 and the cover member 80. In further embodiments, a flexible diaphragm (not shown) (such as, but not limited to, a thin titanium sheet or foil) may be coupled to the inside surface of the cover 80 and sealed around the opening through which the stop 84 extends. The diaphragm will flex to allow the stop to define an adjustable retracted position while also providing sealing functions for inhibiting leakage at the interface between the stop 84 and the cover 80. In other embodiments, after a proper armature position is set, the stop is fixed in place with respect to the cover member, for example, by adhering the stop to the cover member with one or more welds, adhesives or other securing methods.

As shown in FIG. 3, the piston portion 62 of the actuator 58 may extend through the axial channel 38 in the housing 32 toward an outlet at the end of the axial channel 38. The channel 38 may have an inside diameter which is larger than the outside diameter of the piston portion 62. As a result, an annular volume is defined between the piston portion 62 and the wall of the axial channel 38 along the length of the axial channel 38. Infusion medium may flow through the annular volume 82 within the cover 80 to a piston chamber 100 located between the free end of the piston portion 62 and a valve member 102 of a valve assembly 96. In particular embodiments, the radial spacing between the piston portion 62 and the wall of the channel 38 is selected to provide a suitable flow toward the piston chamber 100 to refill the piston chamber 100 (during a return stroke of the piston portion), but small enough to sufficiently inhibit back flow of medium from the piston chamber 100 (during a forward stroke of the piston portion).

The actual radial spacing between the piston portion 62 and the wall of the channel 38 to achieve such results depends, in part, on the overall dimensions of those components, the pressure differentials created in the mechanism, and the viscosity of the infusion medium.

The valve assembly 96 in the embodiment of FIG. 3 may further include the valve member 102 and a valve spring 106. The valve member 102 is located within the outlet chamber 98 and, as shown in FIG. 3, is positioned to close the opening between the axial channel 38 and the outlet chamber 98 when the actuator 58 is in the retracted position. During the forward stroke, the valve member 102 is positioned to open a flow passage between the axial channel 38 and the outlet chamber 98. The valve spring 106 is located within the outlet chamber 98 to support the valve member 102. The spring 106 imparts a spring force on the valve member 102 in the direction toward piston 62 urging the valve member 102 toward a closed position to block the opening between the axial channel 38 and the outlet chamber 98.

The valve member 102 is most often made of generally rigid, biocompatible and infusion medium compatible material, such as, but not limited to, titanium, stainless steel, biocompatible plastic, ceramic, glass, gold, platinum or the like. A layer of silicon rubber or other suitable material may be attached to the rigid valve member material on the surface facing the channel 38 to help seal the opening to channel 38 when the valve member is in the closed position shown in FIG. 3.

The valve spring 106 is most often made of biocompatible and infusion medium compatible material that exhibits a suitable spring force such as, but not limited to, titanium, stainless steel, MP35N cobalt steel or the like. In the illustrated embodiment, the valve spring 106 is a coil spring. In other embodiments, other suitable valve spring configurations may be employed, including, but not limited to, helical, flat, radial, spiral, barrel, hourglass, constant or variable pitch springs or the like.

In the present embodiment, drive mechanism 18 employs electromagnetic and/or mechanical forces to move between a retracted, or first, position and a forward, or second, position, to cause infusion medium to be drawn into and driven out of the pump 18 and the infusion device 10 in a controlled manner.

The position of the actuator 58 in the retracted position may be adjusted by adjusting the position of the stop 84. In one particular embodiment, adjusting the stop 84 includes adjusting a threaded cylindrical member that engages corresponding threads in a stop aperture in the cover member 80 to allow adjustment in a screw-thread manner. An exposed end of the stop 84 may be provided with a tool-engagement depression for allowing engagement by a tool, such as a screw-driver, Allen wrench or the like, from outside of the cover member 80. By engaging and rotating the stop 84 with a suitable tool, the depth that the stop extends into the cover member 80 may be adjusted to adjust the retracted position of the armature portion 60 and therefore the piston 62 in the piston channel 38. In one particular embodiment, adjustments of the stop 84 are made during manufacture. In that embodiment, the adjusted position is set by welding or otherwise adhering the stop 84 in the adjusted position during the manufacture. In other embodiments, the stop 84 is not set and welded during manufacture to allow adjustment of the stop 84 after manufacture.

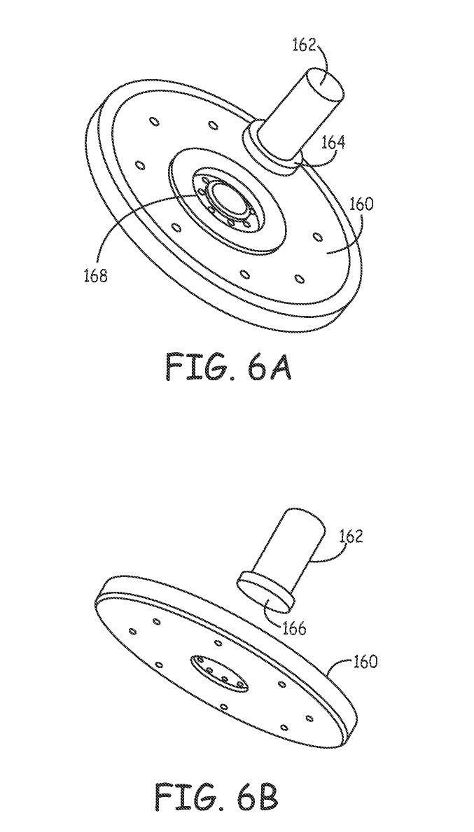

FIGS. 6A and 6B illustrate another embodiment of the actuator 58 of the present invention. As illustrated, the actuator includes a piston 162 and an armature portion 160. The piston 162 may further include a shoulder 164 with a relatively flat engagement surface 166 on the end of the piston 162 designed to contact the armature 160. The armature 160 further includes a contact surface 168 to contact the engagement surface 166 of the piston 162. In the preset embodiment, the contact surface 168 may be a convex shape that distends from the armature 160 a desired amount. Each of the piston 162 and armature portion 160 may be made of substantially one piece and may be formed of any of the materials previously mentioned.

The piston 162 and the armature 160 may be placed into the pump 10 such that the piston 162 resides in the central piston channel 38 in the desired position for pumping. The engagement surface 166 may interact with the convex contact surface 168 at any point along the convex surface. Such a contact may be a point contact. Furthermore, though the armature 160 and the piston 162 are two separate pieces, continuous or near continuous contact between the piston 162 and the armature 160 is achieved because of the force exerted on the piston 162 and the armature 160 by springs 78 and 106 and by the electromagnetic force generated when current is passed through the coil 54. However, the point of contact may vary somewhat without inducing any undesired lateral or rotational forces on the armature 160 or the piston 162 because of the point contact. Moreover, the present embodiment may allow for more degrees of freedom between the armature 160 and the piston 162. Armature 160 may move laterally without causing movement of the piston 162, therefore reducing the strain and wear transferred to the piston 160 and piston channel 38 during the pumping stroke.

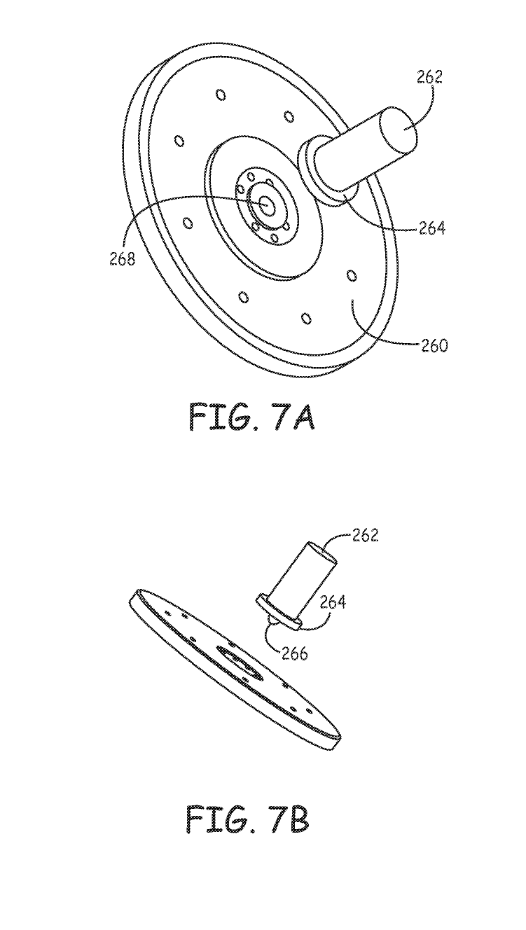

FIGS. 7A and 7B illustrate yet another embodiment of an actuator 58 of the present invention. The actuator 58 includes a piston 262 and an armature 260. The piston 262 may include a shoulder portion 264 and a contact nub 266. The contact nub 266 may be any shape and size that corresponds to a matching socket 268 formed into the armature 260. In the present embodiment, the nub 266 is a generally cone shaped protrusion that is a molded portion of the piston 262. In further embodiments the nub 266 may also be any shape, such as a ball, cone, pyramid, bulge, lump, node, or protuberance, and the socket 268 may be a complimentary shape so as to receive the nub 266. In addition, the piston 262, armature 260, shoulder portion 264, contact nub 266, and socket 268 may be made of any desired material or combination of materials.

The socket 268 may be of a desired shape, depth, and width to engage the contact nub 266, in a manner similar to a ball and socket. Even if the armature 260 moves a little during the pumping stroke the socket 268 on the piston 262 will still engage the contact nub 266 for operation of the pumping stroke. Depending on the amount of friction and the force being applied by the armature 260 during the pumping stroke the socket 268 and contact nub 266, when contacting at an imperfect alignment, may slip the assembly back into the desired orientation or may effectuate the stroke in the non-aligned orientation.

During assembly, the piston 262 may be placed in the central piston channel 38 and the armature 260 placed in a corresponding position to effectuate the desired pumping action. The mating surfaces formed by the nub 266 and the socket 268 may reduce the lateral movement of the piston 262 caused by the armature 260 and therefore reduce strain and wear. Moreover, more lenient manufacturing tolerances may also be allowed. As with the previous embodiment, contact is continuously or almost continuously maintained through the action of the springs 78, 106 and the electromagnetic force due to coil 54.

While at least one exemplary embodiment has been presented in the foregoing detailed description of the invention, it should be appreciated that a vast number of variations exist. It should also be appreciated that the exemplary embodiment or exemplary embodiments are only examples and are not intended to limit the scope, applicability, or configuration of the invention in any way. Rather, the foregoing detailed description will provide those skilled in the art with a convenient road map for implementing exemplary embodiments of the invention, it being understood that various changes may be made in the function and arrangement of elements described in an exemplary embodiment without departing from the scope of the invention as set forth in the appended claims and their legal equivalents.

What is claimed is:

1. An actuator member for a fluid pumping device comprising:
an armature portion; and
a piston portion removably connected to the armature portion.

2. The actuator member of claim 1 further comprising a collar connecting the armature portion and the piston portion.

3. The actuator member of claim 2 wherein the armature portion, the collar, and the piston portion are fittingly connected by corresponding grooves and ribs.

4. The actuator member of claim 3 wherein the armature portion further includes a receiving portion.

5. The actuator member of claim 4 wherein the receiving portion includes a groove and the collar includes a corresponding rib, the collar fitted to the armature by frictionally fitting the rib to the groove.

6. The actuator member of claim 3 wherein the piston portion includes a groove and the collar includes a corresponding rib, the piston fitted to the collar by frictionally fitting the rib to the groove.

7. The apparatus of claim 1 wherein actuator is made of a material with a high magnetic permeability.

8. An actuator for delivering fluid through a piston channel from an inlet to an outlet during a pumping stroke, the actuator comprising:
a piston with an engagement surface, the piston sized and shaped for placement in a piston channel;
an armature with a contact surface, whereby the armature drives the piston through the piston channel during the pumping stroke by the contact surface contacting the engagement surface.

9. The apparatus of claim 8 wherein the contact surface is convex and the engagement surface is relatively flat.

10. The apparatus of claim 8 wherein the contact surface is relatively flat and the engagement surface is convex.

11. The apparatus of claim 8 wherein the contact surface and the engagement surface are complimentary shapes.

12. The apparatus of claim 11 wherein the contact surface is a cone and the engagement surface is a complimentary shaped inverted cone.

13. The apparatus of claim of claim 11 wherein the contact surface is one of a ball or pyramid shape and the engagement surface is a complimentary inverted ball or pyramid shape.

14. The apparatus of claim 8 wherein actuator is made of a material with a high magnetic permeability.

15. The apparatus of claim 8 wherein the actuator is generally rigid.

16. The apparatus of claim 8 wherein the actuator is formed of one or more of ferrous materials or ferritic stainless steel.

17. An apparatus for delivering a fluid, the apparatus comprising:
a housing;
an inlet in the housing for receiving the fluid;
an outlet in the housing for discharging the fluid;
a piston channel within the housing through which the fluid flows from the inlet to the outlet; and
an actuator assembly positioned within the housing and moveable between a first position and a second position, the actuator assembly driving the fluid stored in a piston chamber toward the outlet when the actuator assembly transitions from a retracted position to a forward position, the actuator assembly comprising:
an armature with a contact surface; and
a piston moveable within the piston channel, the piston including an engagement surface that operably interacts with the contact surface of the armature.

18. The apparatus of claim 17 wherein the contact surface is convex.

19. The apparatus of claim 17 wherein the engagement surface is flat.

20. The apparatus of claim 17 wherein the piston further comprises a contact nub.

21. The apparatus of claim 20 wherein the armature further comprises a socket corresponding in shape to the contact nub.

21. The apparatus of claim 20 wherein the contact nub is one or more of the shape of a ball or cone.

22. The apparatus of claim 17 wherein the armature and piston operably interact at a contact point.

23. The apparatus of claim 17 wherein the armature and the piston are in relatively continuous contact during when the actuator assembly transitions from a retracted position to a forward position.
